(19) United States
(12) Patent Application Publication
(10) Pub. No.: US 2012/0098668 A1
(43) Pub. Date: Apr. 26, 2012

(54) INFUSION MONITORING ALARM AND METHOD FOR MONITORING AND ALARMING FOR INTRAVENOUS INFUSION

(75) Inventor: Yimin LI, Shenzen (CN)

(73) Assignee: Peng Chen

(21) Appl. No.: 13/279,136

(22) Filed: Oct. 21, 2011

(30) Foreign Application Priority Data
Oct. 22, 2010 (CN) 201020572670.8
May 20, 2011 (CN) 201120163741.3

(51) Int. Cl. G08B 21/00

(52) U.S. Cl. 340/657

(57) ABSTRACT

An infusion monitoring alarm is attached to an infusion tube between a solution bottle and a dripping chamber, including a clamping piece; at least two polar plates forming a capacitor; a capacitance measurement unit configured to measure a capacitance of the capacitor; a micro control unit configured to collect data from the capacitance measure unit and generate a warning signal when the capacitance value of the capacitor decreases beyond a predetermined threshold value and a power supply circuit configured to provide electricity. A method for monitoring and alarming for intravenous infusion includes developing a baseline value of a capacitor at an initiation of the infusion procedure; determining a current value of a capacitor and comparing the current value of a capacitor with the baseline value; and sending out a signal for alarming when current value of the capacitor decreases beyond a predetermined percentage of the baseline value of the capacitor.

### CROSS-REFERENCE TO RELATED APPLICATION

This application claims the right of priority based on Chinese applications no. 201020572670.8 and no. 201120163741.3.

### BACKGROUND

1. Technical Field

The present invention relates to the field of electronic medical technology. More particularly, the present invention relates to a device and method for monitoring and alarming for intravenous infusion.

2. Related Art

Intravenous infusions are widely used in current medical practices. Use of disposable intravenous infusion devices in elderly, young, military, and seriously ill patient often require long hours of continuous monitoring by caregivers. Just before the infusion solution is depleted, the caregiver will promptly notify a medical staff person to inform that that the solution needs to be changed or that the needle needs to be removed. Time is off the essence in order to prevent complications such as blood backflow or air bubbles trapped in the infusion tube. The continual monitoring of the infusion solution level by people is extremely inconvenient and burdensome for the caregiver. There is also the danger that the caregiver may neglect their duty, especially during the night when the caregiver may be drowsy. To help combat this problem, a variety of alarm systems have been developed. For example, an infusion alarm system uses spring balances to monitor the weight of the solution and electronic circuits to measure properties such as impedance, light attenuation, or light refraction. However, these systems have drawbacks such as low accuracy, jamming potential, high power consumption, solution degradation, etc. As another example, infrared light is used to monitor the solution. However, light might irradiate the medical solution, which makes the alarm system not suitable to light-sensitive medical solution. These, and other drawbacks make the practical value of medical equipment, especially electronic medical equipment, lower than desired.

### BRIEF SUMMARY

The present application provides an infusion monitoring alarm, attached to an infusion tube between a solution bottle and a dripping chamber, comprising: a clamping piece; at least two polar plates forming a capacitor, wherein the at least two polar plates are spaced to accommodate the infusion tube; a capacitance measurement unit configured to measure a capacitance of the capacitor, wherein the capacitance measurement unit is electrically connected to the capacitor and the capacitance measurement unit is an integrated circuit specially designed for capacitance measurement; a micro control unit (MCU) configured to collect data from the capacitance measure unit and generate a warning signal when the capacitance value of the capacitor decreases beyond a predetermined threshold value, wherein the MCU is electrically connected to the capacitance measurement unit; a power supply circuit configured to provide electricity, the power supply circuit including a power supply and a micro swift; and an alarm piece configured to send an alarm based on the warning signal, wherein the alarm piece is electrically connected to the MCU.

The present application also provide an infusion monitoring alarm, comprising: a clamping piece; at least two polar plates forming a capacitor, wherein the at least two polar plates are spaced to accommodate an infusion tube; a capacitance measurement unit configured to measure a capacitance of the capacitor, wherein the capacitance measurement unit is electrically connected to the capacitor; a MCU configured to collect data from the capacitance measure unit and generate a warning signal when the capacitance value of the capacitor decreases beyond a predetermined threshold value, wherein the MCU is electrically connected to the capacitance measurement unit; a power supply circuit configured to provide electricity; and an alarm piece configured to send an alarm based on the warning signal, wherein the alarm piece is electrically connected to the MCU.

The present application also provide a method for monitoring and alarming for intravenous infusion, comprising the steps of: developing a baseline value of a capacitor at an initiation of the infusion procedure; determining a current value of a capacitor and comparing the current value of a capacitor with the baseline value; and sending out a signal for alarming when current value of the capacitor decreases beyond a predetermined percentage of the baseline value of the capacitor.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is a schematic illustration of one embodiment of an infusion monitoring alarm.

FIG. 2 is a schematic illustration of another embodiment of an infusion monitoring alarm.

FIG. 3 is a schematic illustration of one embodiment of a circuit board.

FIG. 4 is a circuit schematic illustration corresponding to FIG. 3.

FIG. 5 illustrates a first embodiment of the electrical connections with the capacitance measurement unit being an integrated circuit.

FIG. 6 illustrates a second embodiment of the electrical connections with the capacitance measurement unit being dedicated circuit.

FIG. 7 illustrates a third embodiment of the electrical connections with the capacitance measurement unit being dedicated circuit.

FIG. 8 illustrates an embodiment of the micro switch.

FIG. 9 illustrates an embodiment of the method.

FIG. 10a-10c indicate the principle of the device.

### DETAILED DESCRIPTION

Hereinafter, the present invention will be described in detail with reference to the accompanying drawing regarding the area division principle, selection of measuring point, evaluation standard and construction of the system. It should be understood that drawing does not limit the scope of the present invention.

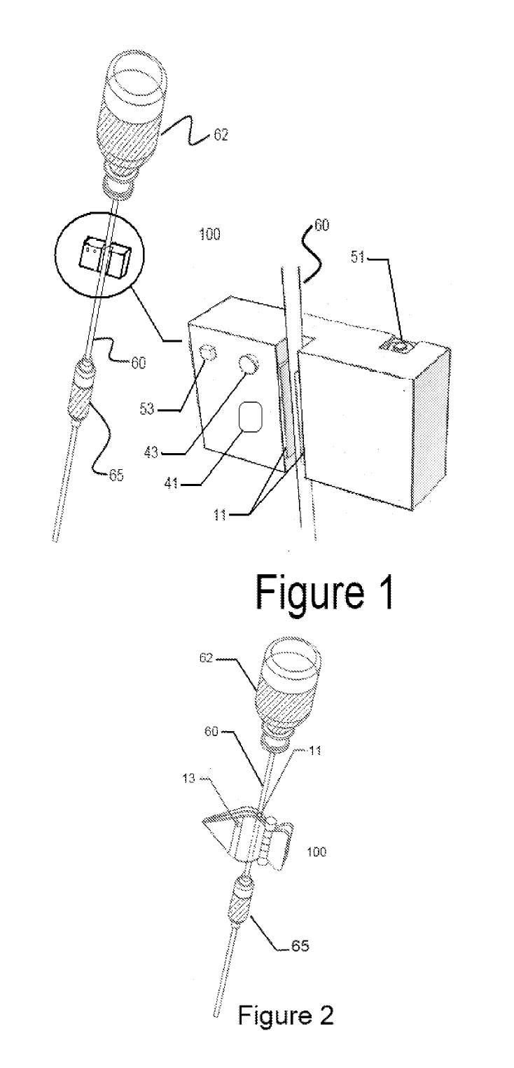

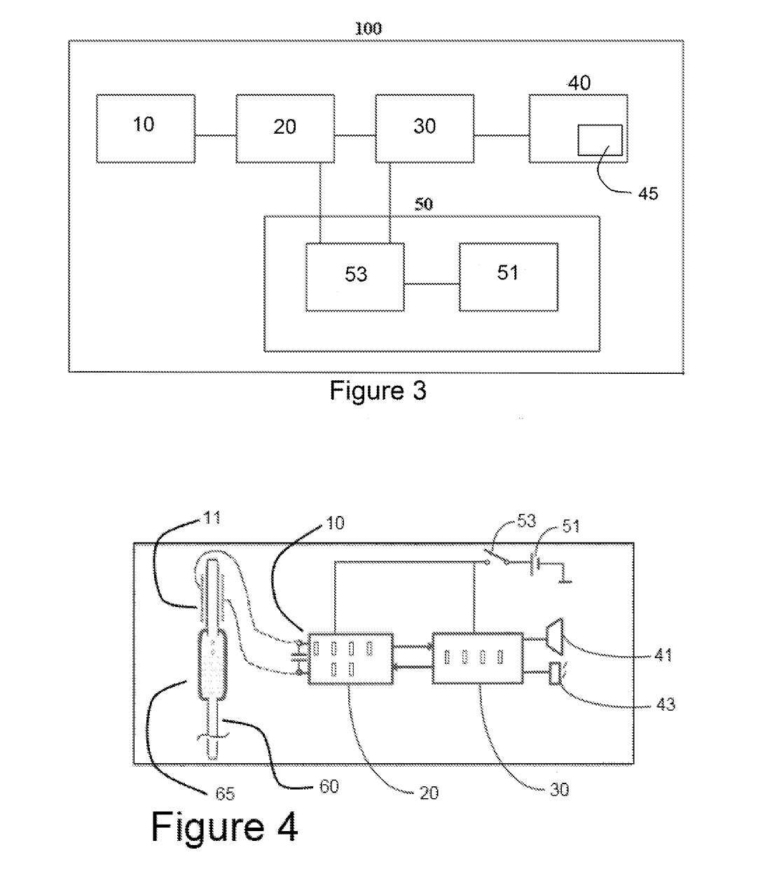

Please refer to the embodiments shown in FIGS. 1-4. FIG. 1 is a schematic illustration of one embodiment of an infusion monitoring alarm. FIG. 2 is a schematic illustration of another embodiment of an infusion monitoring alarm. FIG. 3 is a schematic illustration of one embodiment of a circuit board. FIG. 4 is a circuit schematic illustration corresponding to FIG. 3. An infusion monitoring alarm may include a clamping piece 100 (shown in FIG. 1 and FIG. 2), an alarming circuit (shown in FIG. 3 and FIG. 4) and an alarm piece 40 (shown in FIG. 3 and FIG. 4) for alarming. The alarming circuit and the alarm piece may be located either within or attaching to the clamping piece 100. The clamping piece 100 may be attached to an infusion tube 60 between an infusion bottle (bag) 62 and a dripping chamber 65. The shape of the clamping piece can take any number of forms, including, for example, a rectangular box (as shown in FIG. 1), heart (as shown in FIG. 2), butterfly, cartoon, character, or other desirable shape. The clamping piece 100 can also have changeable face plates to suit the patient's preference and help increase their comfort whilst receiving treatment. The infusion tube 60 can be made of any material and have any sized diameter.

As shown in FIG. 1, the clamping piece may include an opening in which at least two polar plates 11 are placed. The opening can be a channel, spring clip, caliper or other structures achieving the similar function. The polar plates 11 can be made of any metal, though it is preferred that they are made of copper, a copper alloy or stainless steel. The polar plates 11, can also take a variety of different shapes. Such shape includes, but is not limited to, flat, arched, rectangular, or oblong. It is preferred that the polar plates 11 are semi-circular, though the size of the polar plates is not limited. It is further preferred that the radius of the semi-circular plates matches the radius of the infusion tube 60, though the size of the plates is not limited. The clamping piece 100 may have one or more positioning pieces 13. The positioning piece 13 may be either an adjustable or fixed-size piece that prevents extremely deformation of the infusion tube 60. It is desirable to have the diameter of the positioning piece 13 match the diameter of the infusion tube 60. The positioning piece 13 can be a round bar or a convex column, but is not limited to such shapes.

As shown in FIG. 3, the alarming circuit may contain a capacitor 10, a capacitance measurement unit 20 for measuring the capacitance, a micro control unit (MCU) 30 for controlling the system and a power supply circuit 50 for providing power. The capacitance measurement unit 20 may be electrically connected to the capacitor 10. The MCU 30 may be electrically connected to the capacitance measure measurement unit 20. The alarm piece 40 may be electrically connected to the MCU 30. The power supply circuit 50 may be electrically connected to the capacitance measurement unit 29 and the MCU 30. In some embodiments, the power supply circuit 50 might also be electrically connected to the alarm piece 40.

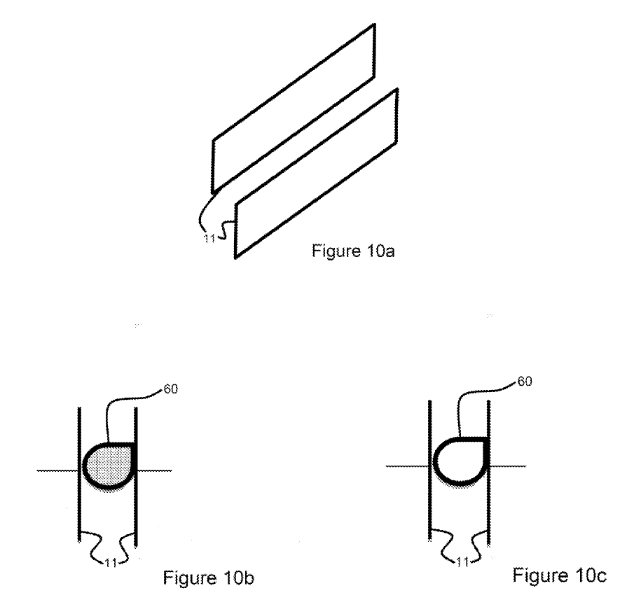

The infusion monitoring alarm functions based on the principle below. Please refer to FIG. 10a-10c, which indicate the principle of the device. A classic capacitor is composed of two metal polar plates 11, the shape, area, and distance between the plates being fixed and known. If two plates are flat and symmetrical, with no filling substance between the plates, the capacitance can be calculated by the following equation:

$$C = ɛ * ( \frac{S}{d} )$$

where C is capacitance, E is the dielectric constant of the capacitor, s is the area of the metal plates, and d is the distance between the two metal plates. Assuming the distance d and the area of the metal plates s are fixed, the value of capacitance C decreases when the value of dielectric constant ∈ decreases. It is known that the value of dielectric constant E of air is smaller than that of any liquid. Therefore, the value of capacitance C decreases when the solution becomes depleted. In this application, two polar plates are put respectively on both sides of the tube 60 between the infusion bottle (bag) 62 and the dripping chamber 65. As a result, the distance d and the area of the metal plates s are fixed. When the value of capacitance C decreases during the measurement, it indicates that the solution in tube becomes less and less. Therefore, when the value of capacitance C measured by the capacitance measurement unit 20 decreases to or over a predetermined percentage, it indicates that the solution may become almost depleted. Under that situation, MCU 30 may send the signal to the alarm piece 40.

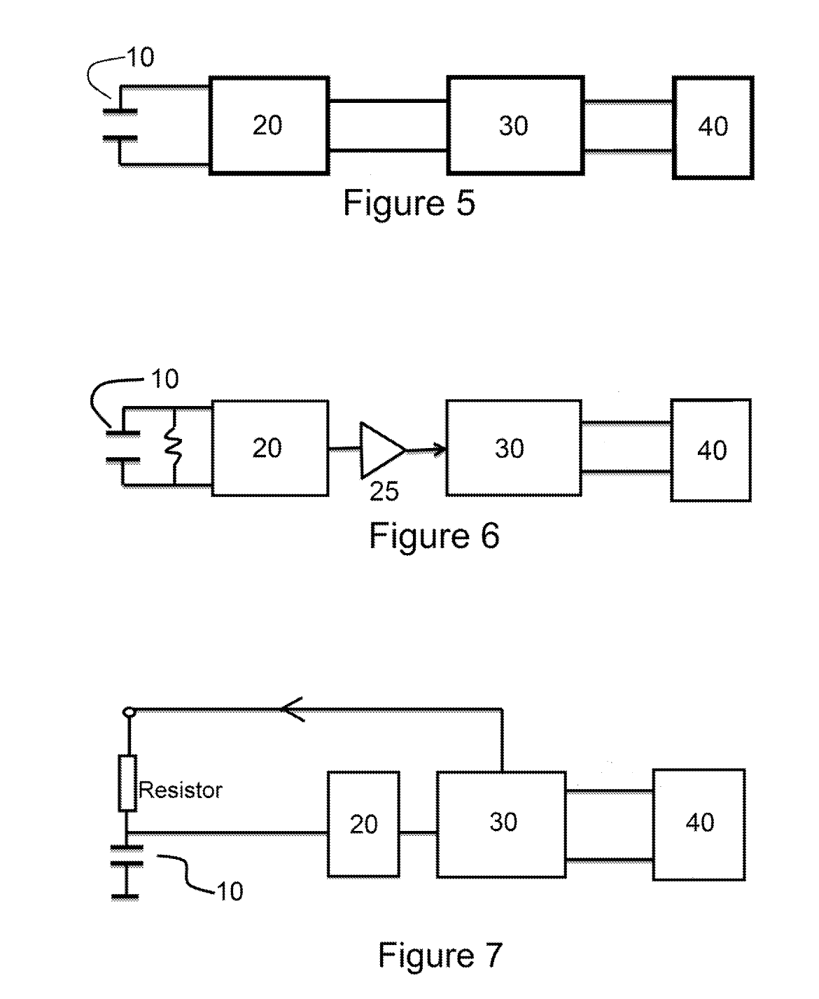

The capacitance measurement unit 20 can either be an integrated or a dedicated circuit. FIG. 5 illustrates a first embodiment of the electrical connection with the capacitance measurement unit 20 being an integrated circuit. FIG. 6 illustrates a second embodiment of the electrical connection with the capacitance measurement unit 20 being dedicated circuit. FIG. 7 illustrates a third embodiment of the electrical connection with the capacitance measurement unit 20 being dedicated circuit.

As shown in FIG. 5, the capacitance measurement unit 20 may be a capacitance-specific integrated circuit (IC), with the polar plates 11 functioning as the capacitor. The capacitance measurement unit 20 can be a special IC exclusively designed for capacitance measurement, for example, some of the newly developed IC by ANALOG DEVICES®. The IC for capacitance measurement can output the capacitance value using the serial data format. The serial data may be transmitted to the MCU 30. The MCU 30 can create a baseline measurement by measuring the capacitance at a certain time, for example, when a medical solution is flowing through the infusion tube 60, either by a single measurement or average of multiple measurements. The MCU 30 periodically collects data and then compares it to the baseline value. A certain number of collected data points may be averaged before comparing the average to the measured baseline value. Once the MCU 30 determines that the value of capacitance has decreased to a predetermined threshold when compared to the baseline value, it will output a controlling signal, such as a square wave, via I/O port or other means to an alarm piece 40. In one embodiment, the predetermined threshold is 90% from the baseline value.

As shown in FIG. 6, the capacitance measurement unit 20 is an LC oscillator (resonant) circuit, with the polar plates 11 functioning as the capacitor. Signals generated by the LC oscillator circuit are amplified by an amplifier 25 and transmitted to the MCU 30. The MCU 30 is electrically connected to an alarm piece 40, either by an I/O port or other means. The frequency of the signals generated by an LC oscillator is determined by the following equation:

$$F = \frac{1}{2 π \sqrt{LC}}$$

where F is the frequency in hertz, L is the inductance in henries, and C capacitance in farads. The MCU 30 acts as a frequency counter to record the frequency of the signals sent from the LC oscillator. It was discovered by experimentation that the inductance L in this embodiment would remain the same. Therefore, the frequency of the signal is based only on the measured capacitance. For example, if the capacitance is reduced by one fourth (¼), the measured frequency would increase two-fold. A baseline frequency is determined. The baseline can be calculated when a medical solution is flowing through the infusion tube 60. The MCU 30 then compares this baseline to subsequent frequency measurements. The comparison can be with a single measurement or group of measurements. When the MCU 30 determines that the frequency has crossed over a predetermined threshold amount, it will send a signal to the alarm piece. One such predetermined threshold amount of the frequency could be when the corresponding capacitance has been reduced to or over 90% from the capacitance corresponding to the baseline frequency.

As shown in FIG. 7, the capacitance measurement unit 20 is a resistor-capacitor (RC) circuit. The resistor R is electrically connected to the capacitor in series. The resistor R is also electrically connected to the MCU 30. The capacitor and resistor are electrically connected to the Analog to Digital converter (A/D converter). The A/D converter is electrically connected to the MCU 30 and the MCU 30 is electrically connected to the alarm piece 40. The resistance of the resistor R is known. The capacitance measurement unit 20 measures the voltage at a certain time, such as when a medical solution is flowing through the infusion tube 60. The capacitance measurement unit 20 then measures the voltage of the system until it is determined that the current voltage is equal to half of the voltage set as the baseline measurement. At this time and afterwards, the capacitance of the system can be determined using the following equation:

$${V}_{c} = {V}_{0} * ( 1 - {e}^{\frac{- t}{RC}} )$$

where V_{c} is the current voltage at time t, V₀ is the voltage equal to the baseline voltage, R is the resistance, and C is the capacitance. The capacitance measurement unit 20 continues to measure the current voltage and sends the data to the MCU 30 until the MCU 30 determines the calculated capacitance is below a predetermined threshold. In one embodiment, the predetermined threshold is a 90% from the baseline value. The baseline value may be measured at an initial time. When the MCU 30 determines that the threshold value has been crossed, it generates and sends a signal to the alarm piece 40. The generated signal can be sent to the alarm piece 40 via an I/O port.

The MCU 30 may be able to collect the data from the capacitance measurement unit 20 and send the signal to the alarm piece 40 as mentioned above. The MCU 30 can be any type of electronic circuit but is preferably a programmable intermittent working microprocessor. The MCU 30 can be programmed to send a signal after a certain period of time during the infusion process or upon the occurrence of a specified event or group of events. An example of such an event is the low level of the infusion solution. As stated above, the MCU 30 may send the signal to the alarming piece 40 when the capacitance value of the capacitor decreases beyond a predetermined threshold value, for example, 90% of the baseline capacitance value.

The other example of the event for the MCU 30 to send out a signal is infusion irregularities. The infusion irregularities may include, but not limited to, the irregularity of infusion rate, a blockage in the infusion tube 60 and air bubbles trapped in the infusion tube 60. During the regular infusion process, the medical solution drops constantly. Consequently, the medical solution flows regularly in the infusion tube between the infusion bottle (bag) 62 and the dripping chamber 65. It is known that the dielectric constant of the same liquid is “slightly different” in stationary and mobile states. Therefore, the capacitance measurement unit 20 may be able to obtain a series data of capacitor having “pulse” character. If the MCU 30 monitors the capacitance at a high enough frequency, the frequency of “pulses” can reflect the infusion rate. When MCU 30 determines the “pulses” become irregular, it may signal the alarm piece 40 to alert the medical staff of the irregularity. Furthermore, when the average capacitance is within a range of the baseline capacitance value and also the “pulse” has stopped, it indicates that the infusion tube is still filled with liquid but the dripping has been stopped or become very slow drip rate. This situation may happen when the medical solution is injected into surrounding tissues (e.g., muscle) instead of the blood stream or other similar infusion blockages have occurred. Corresponding warnings can be issued by the alarm piece 40. Air bubbles passing through the infusion tube 60 can also be determined by the MCU 30. The MCU 30 may be programmed to search for such irregularities by monitoring the changes of the measured capacitance of the medical solution passing through the infusion tube 60. If the MCU 30 determines that the measured drop in capacitance has crossed the threshold for more than a predetermined period of time, the MCU 30 may send a signal to the alarm piece 40 to alert the medical staff that an irregularity has occurred. This system can allow the medical staff to monitor the infusion rate to ensure proper treatment.

The MCU 30 can also be programmed with a wake-up and standby to control the capacitance measurement unit 20 periodically working, which can significantly help conserve battery life. The wake-up period can be programmed to any time. For example, the MCU 30 wakes up—either every other minute or every other ten second. The MCU 30 can be further programmed to work for a specified period of time when it wakes up. The specified period is entirely adjustable. But it is preferable that when the MCU 30 wakes up, it works for 100 ms each time. The function above helps to conserve battery life. As a result, the monitoring alarm can consume electricity to several milliampere for about twenty hours. It is possible to have non-continuous measurements, including wake up functions, without the fear that the medical solution will no longer be present in the infusion tube 60 since even after the medical solution has been depleted from the storage container or bag, the some medical solution remains in the dripping chamber 65. This residual solution will take at least a few more minutes to pass thorough the infusion tube 60.

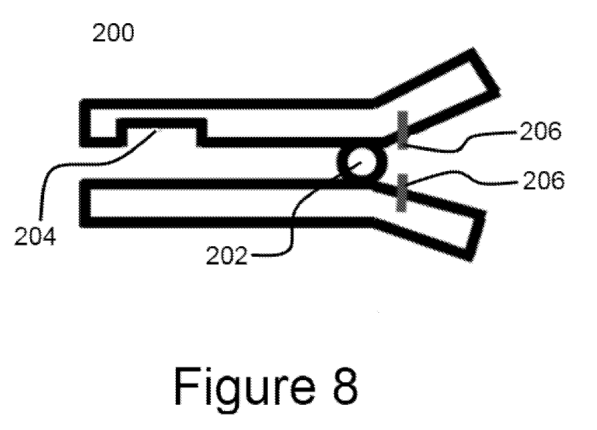

The alarming circuit of the infusion monitoring alarm may further include a power supply circuit 50. The power supply circuit 50 may be electrically connected to the capacitance measurement unit 20 and/or the MCU 30. The electrical connections may be in such a way to allow the power supply circuit 50 to provide electricity to any component it is electrically connected to. The power supply circuit 50 may further include a power supply 51 and a micro switch 53. The power supply 51 can be any source of power including, but not limited to, a solar panel or a button cell battery. The micro switch can be any type of switch including, but not limited to a touch button switch or a mechanical contact micro switch, such as micro switch 53 shown in FIG. 1. Another potential embodiment of the micro switch 53 may be a clamping micro switch as shown in FIG. 8. The clamping micro switch is located within the clamping piece 100 and is triggered when the infusion tube 60 is placed within the clamp piece 200. When the infusion tube 60 is placed within the positioning piece 13, foots of the clamping pieces 100 pivot about a fulcrum 202 to accommodate the infusion tube 60 within the clamping groove 204. The foots of the clamping pieces 100 contact each other through the contacting pieces 206, located at an end of the clamp 200 opposite where the infusion tube 60 is located. The contact between the foots of the contact pieces allow for a circuit to be completed, thereby allowing power to flow through the system. The micro switch 53 can be the main switch for controlling power to the entire system.

The infusion monitoring alarm may also contain an alarm piece 40. The alarm piece 40 may contain a notifying portion, alerting users of a certain event mentioned above during the infusing process. The notifying portion that alerts the user of any pre-specified event can optionally take any form, including, but not limited to, a buzzer, lighting device such as a light emitting diode, wired network, or a wireless network. The MCU 30 can be programmed to activate any or all of the notifying portions after the occurrence of any event or a specified time after the occurrence of the event. The notifying portion(s) can also be silenced or deactivated either after a certain amount of time, upon an event, or during the entire infusion process. When and how the alarm piece 40 is activated is based on the program of the MCU 30.

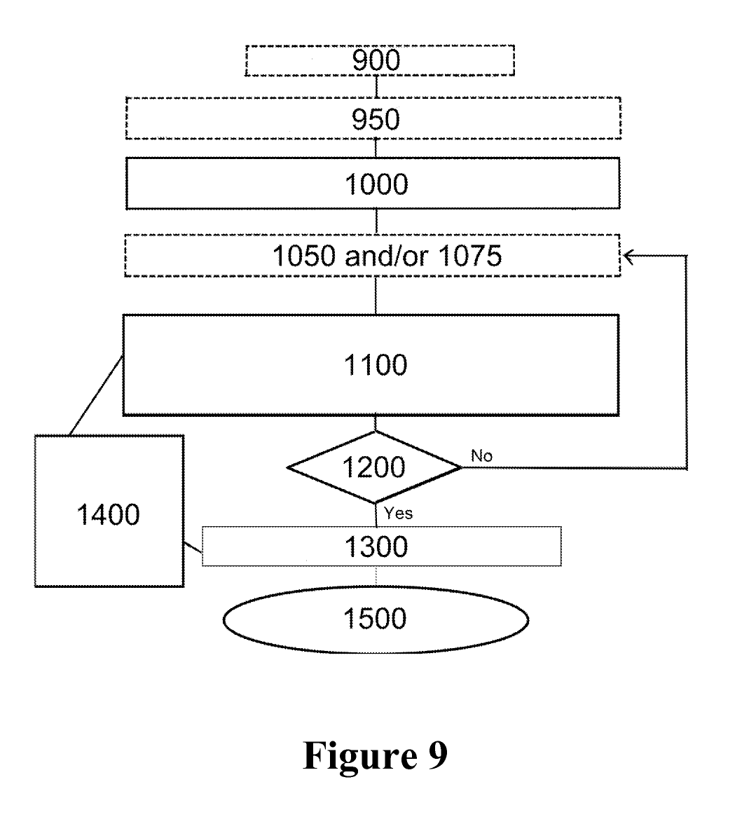

Utilizing the above device or a different device, an infusion procedure can be monitored. The aspects of the infusion procedure that can be monitored include, but are not limited to, infusion rate and depletion level. An embodiment of the method is depicted in FIG. 9. Since the steps can be performed in any order, the order of the steps shown in FIG. 9 is used merely for references for discussing the particular embodiment.

Step 1000 is the development of a baseline value. The baseline value can be calculated at any point during or before the infusion procedure. For example, it may be calculated at the initiation of the infusion procedure or at some fixed time after the procedure has begun. In this specific embodiment, the baseline value is calculated by measuring the capacitance once the medical solution has begun to flow through the infusion tube. The capacitance is measured using two metal polar plates surrounding the infusion tube. Other measurements include voltage and frequency of a signal. The baseline can be established by taking a single capacitance measurement or by performing a function on multiple capacitance measurements, such as mean, median, or standard deviation.

Step 1100 is a subsequent measurement to determine the current capacitance. In the particular embodiment, the measurements are capacitance measurements. These measurements are used to determine the current capacitance of the infusion tube. The current capacitance can be determined either by a single measurement or by performing a function on multiple measurements, such as mean or median. The capacitance can be measured utilizing numerous devices and techniques. For example, the capacitance may be measured using a two or more metal polar plates. The plates can then be connected to a capacitance measurement unit, which can either be a dedicated or an integrated circuit. As an integrated circuit, the capacitance measurement unit may be electrically connected to MCU 30. The MCU 30 will analyze the capacitance measured using across the two metal polar plates. The capacitance measurement unit 20 can be a special IC exclusively designed for capacitance measurement, for example, some of the newly developed IC by ANALOG DEVICES®. As a dedicated circuit, the capacitance measurement unit may be an RC circuit. The RC circuit will measure the current voltage of the system. Utilizing the voltage calculated whilst performing the baseline determination step, the MCU 30 can calculate the current capacitance of the infusion tube. Another form of dedicated circuit the capacitance measurement unit may take is an LC oscillator circuit. The frequency of the signal can be used to develop a baseline capacitance. Then a measured change in the frequency would indicate a change in capacitance. For example, a two-fold change in capacitance would indicate that the capacitance of the system would be one-fourth (¼).

The current measured capacitance is compared to the baseline value determined in step 1000, an embodiment of which is indicated by step 1200. The comparison determines, either through an MCU 30 or other device or method, if current measured capacitance decreases to or over a predetermined threshold. For example, but not limited to such example, the threshold can be set to be 90% of the initial baseline value. The threshold could be set to a much lower or greater percentage of the measured capacitance. Alternatively, the threshold could be set to a predefined capacitance measurement in step 1000. Thus the comparison will be made to determine if the current measured capacitance is lower than the baseline value determined in step 1000. Once a comparison to the threshold is made and the comparison determines that the threshold has not been reached, the system will return to system 1100 to re-measure the current capacitance. If the comparison determines that the current measured capacitance reaches or becomes lower than 90% of the threshold, the system will send a signal to activate an alarm unit. The signal can be sent from a device such as the MCU 30, but is not limited to this device. The signal can be any type of signal, such as a square wave digital signal, an analog signal, or other signal.

Step 1300 depicts an embodiment of the alarm step. The alarm step of this embodiment is activated by a signal when a device such as the MCU 30 determines that the current measured capacitance reaches or becomes lower than the threshold. The alarm step can be used to alert a medical practitioner, such as a doctor or nurse, or other persons, such as the patient or family member, that the infusion procedure is in need of attention. The types of alarm include, but are not limited to, buzzers, light emitting diodes, wired signals, or wireless signals. The wired or wireless signals may alert a practitioner or other person who is not in the immediate vicinity of the infusion procedure. The practitioner or other person may be a location such as at home or in a nurse's station. One or more alarms may be activated, either in unison or with a certain sequence. The sequence can be, for example, alerting a practitioner or user utilizing a first type of alarm, and, if the practitioner or other person does not deactivate the alarm, a second alarm may attempt to alert the practitioner or other person. The process is complete when the alarm is deactivated, step 1500, either by a person, such as a medical practitioner or other person, or automatically, such as by the program implemented by the MCU 30.

In addition to the above steps, other steps may be included to assist with proper functioning and power-saving abilities of the system. In example step 900, an MCU 30, if present and after being supplied power, may initiate the whole system and configure the parameters of the other integrated circuits, if present. The alarm system may be tested to ensure working status and to ensure that enough voltage is supplied to it. An embodiment of the alarm testing step includes an MCU 30 testing the notifying portion 45, such as an LED and/or buzzer, to ensure proper working status and to test the power supply circuit 50 and the power supply 51 to ensure proper voltage supply.

In the present embodiment, step 950 is a period of waiting to ensure that the system and device are in a steady state. This waiting period can be for any amount of time, including a few seconds and a few minutes. It is preferred that the waiting period is a few seconds, for example 5 seconds. During the waiting period, the system and device should be in an equilibrium state. This period of equilibrium to ensure the system is in a steady state helps ensure proper functioning of the device and system during treatment.

In the embodiment, there is the additional, optional step of 1050. In this step, the device enters a power saving state. One of the goals of this device is to help preserve the battery life of device, when the device is running on internal power. One way the device may enter a power saving state is at the direction of the MCU 30. The MCU 30 can be programmed to control itself, and optionally other program controlled integrated circuits, to enter a standby mode. It should be noted that other methods of entering power saving states may be employed. It should be further noted that power saving states may not always be desirable and are treatment dependent. For example, if there is fear that an air bubble may form in the infusion tube and the device has been set up to employ the air bubble detection mechanism described above, the device may continuously take measurements and would not enter a power saving state. Under normal treatment circumstances, a power saving function may be desirable. Step 1050 may occur at various times during the method. For example, it preferably occurs after the baseline value has been determined and if the procedure protocol allows for the device to enter a power saving state. It is further preferable for the method to cycle back to step 1050 if the current capacitance measurement determines that the current capacitance has not met or exceeded the threshold value, assuming the treatment protocol allows for the system to enter a power saving state.

If step 1050 is utilized, it is optional, though preferable, to further employ 1075. Step 1075 the whole system wakes up from the power saving state. Every component can wake up simultaneously, in steps, or in groups. It is preferred that the MCU 30 first wake up and then signal the other components that entered standby to wake up. The amount of time the various components entered a standby state, or other power saving state, can be predetermined by a program. The program can optionally be implemented in the MCU 30.

Step 1400 may optionally be employed, and is currently employed in the embodiment depicted in FIG. 9. Step 1400 describes a step that may help ensure proper functioning of the device and system. This can be accomplished by monitoring the state of the battery. For example, the voltage of the battery may be monitored. Such battery monitoring functions may be initiated by an independent program or by the MCU 30. The MCU 30 may have its own program to determine when and how to monitor the state of the battery. For example, the MCU 30 may monitor the battery during the concurrently with measurement cycle. If the battery state is determined to be non-compliant with the pre-set standards, such as the voltage is too low to meet the requirements, an alarm may be activated. The alarm may be activated by the MCU 30 sending a signal to the alarm piece 40. It is preferable, though not required, that the activated alarm, such as an LED, buzzer, wired network, or wireless network, differ from the alarm generated for an issue dealing strictly with the infusion procedure. For example, the differing alarm could be a continuous sound from a buzzer.

The above description is given by way of example, and not limitation. Given the above disclosure, one skilled in the art could devise variations that are within the scope and spirit of the invention disclosed herein, including configurations ways of the recessed portions and materials and/or designs of the attaching structures. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. An infusion monitoring alarm, attached to an infusion tube between a solution bottle and a dripping chamber, comprising:
a clamping piece;
at least two polar plates forming a capacitor, wherein the at least two polar plates are spaced to accommodate the infusion tube;
a capacitance measurement unit configured to measure a capacitance of the capacitor, wherein the capacitance measurement unit is electrically connected to the capacitor and the capacitance measurement unit is an integrated circuit specially designed for capacitance measurement;
a micro control unit (MCU) configured to collect data from the capacitance measure unit and generate a warning signal when the capacitance value of the capacitor decreases beyond a predetermined threshold value, wherein the MCU is electrically connected to the capacitance measurement unit;
a power supply circuit configured to provide electricity, the power supply circuit including a power supply and a micro swift; and
an alarm piece configured to send an alarm based on the warning signal, wherein the alarm piece is electrically connected to the MCU.

2. The infusion monitoring alarm described in claim 1, wherein the predetermined threshold value is ninety percent of a baseline capacitance value.

3. The infusion monitoring alarm described in claim 1, wherein the power supply is a button cell battery.

4. The infusion monitoring alarm described in claim 1, wherein the MCU is programmed with a wake-up function and is programmed to periodically operate to help conserve battery life.

5. The infusion monitoring alarm described in claim 1, wherein the alarm piece is a signaling device selected from the group consisting of a buzzer, light-emitting diode, wired network, and a wireless network.

6. An infusion monitoring alarm, comprising:
a clamping piece;
at least two polar plates forming a capacitor, wherein the at least two polar plates are spaced to accommodate an infusion tube;
a capacitance measurement unit configured to measure a capacitance of the capacitor, wherein the capacitance measurement unit is electrically connected to the capacitor;
a micro control unit (MCU) configured to collect data from the capacitance measure unit and generate a warning signal when the capacitance value of the capacitor decreases beyond a predetermined threshold value, wherein the MCU is electrically connected to the capacitance measurement unit;
a power supply circuit configured to provide electricity; and
an alarm piece configured to send an alarm based on the warning signal, wherein the alarm piece is electrically connected to the MCU.

7. The infusion monitoring alarm described in claim 6, wherein the predetermined threshold value is ninety percent of a baseline capacitance value.

8. The infusion monitoring alarm described in claim 6, wherein the capacitance measurement unit is an integrated circuit specially designed for capacitance measurement.

9. The infusion monitoring alarm described in claim 6, wherein the capacitance measurement unit is a resonant (LC) circuit and the capacitance is measured by equation:
$$F = \frac{1}{2 π \sqrt{LC}} ,$$
where F is the frequency in hertz, L is the inductance in henries, and C capacitance in farads.

10. The infusion monitoring alarm described in claim 6, wherein the capacitance measurement unit is a resistor-capacitor (RC) charging circuit and the capacitance is measured by equation:
$${V}_{c} = {V}_{0} * ( 1 - {e}^{\frac{- t}{RC}} ) ,$$
where V_{c} is the current voltage at time t, V₀ is the voltage equal to the baseline voltage, R is the resistance, and C is the capacitance.

11. The infusion monitoring alarm described in claim 6, wherein the alarming circuit and the alarm piece are located within the clamping piece.

12. The infusion monitoring alarm described in claim 6, wherein the MCU generates a warning signal when an infusion irregularity occurs and the infusion irregularity selects from the group consisting of an irregularity of infusion rate, a blockage in the infusion tube and air bubbles trapped in the infusion tube.

13. The infusion monitoring alarm described in claim 6, wherein the MCU is programmed with a wake-up function and is programmed to periodically operate to help conserve battery life.

14. The infusion monitoring alarm described in claim 6, wherein the power supply circuit further includes a power supply and a micro switch configured to control the power supply.

15. The infusion monitoring alarm described in claim 14, wherein the power supply is a button cell battery.

16. The infusion monitoring alarm described in claim 6, wherein the alarm piece is a signaling device selected from the group consisting of a buzzer, light-emitting diode, wired network, and a wireless network.

17. The infusion monitoring alarm described in claim 6, wherein the distance between the two plates matches a diameter of the infusion tube.

18. The infusion monitoring alarm described in claim 6, wherein the shape of the clamping piece selects from the group consisting of a rectangular box, heart, and butterfly.

19. A method for monitoring and alarming for intravenous infusion, comprising the steps of:
developing a baseline value of a capacitor at an initiation of the infusion procedure;
determining a current value of a capacitor and comparing the current value of a capacitor with the baseline value; and
sending out a signal for alarming when current value of the capacitor decreases beyond a predetermined percentage of the baseline value of the capacitor.

20. The method described in claim 19, wherein the value of a capacitor is calculated by an integrated circuit specially designed for capacitance measurement.
